(19) United States
(12) Patent Application Publication
(10) Pub. No.: US 2007/0053930 A1
(43) Pub. Date: Mar. 8, 2007

(54) Combination therapy for treatment of high cholesterol

(75) Inventor: Chad Brown, Newport Beach, CA (US)

Correspondence Address: HELLER EHRMAN WHITE & MCAULIFFE LLP, 1717 RHODE ISLAND AVE, NW, WASHINGTON, DC, 20036-3001, US

(73) Assignee: BEBAAS, INC., Newport Beach, CA (US)

(21) Appl. No.: 11/219,793

(22) Filed: Sep. 7, 2005

(51) Int. Cl. A61K 36/16; A61K 9/48; A61K 36/06; A61K 31/366; A61K 31/22

(52) U.S. Cl. 424/195.16; 424/752; 514/460; 514/548; 424/451

(57) ABSTRACT

Compositions and convenient methods for the delivery of a therapeutic combination of statins such as mevinolin, with niacin and ginkgo to patients with high blood cholesterol are provided.

### BACKGROUND OF THE INVENTION

High blood cholesterol is a major risk factor for heart disease. Heart disease is the number one killer of women and men in the United States and, each year, more than a million Americans have heart attacks, and about a half million people die from heart disease. Blood cholesterol consists of three components: triglycerides, high density lipoprotein (HDL), often referred to as “good” cholesterol, and low density lipoprotein (LDL) often referred to as “bad” cholesterol. LDL lipoprotein deposits cholesterol on the artery walls, causing the formation of a hard, thick substance called cholesterol plaque. Over time, cholesterol plaque causes thickening of the artery walls and narrowing of the arteries, a process called atherosclerosis.

Apart from therapeutic lifestyle changes such as a diet, physical activity and weight management, several drugs or drug combinations are available to lower the bad cholesterol and/or raise the good cholesterol levels. Statin drugs such as lovastatin are effective in therapy as they lower the LDL levels and are safe for most people. Niacin (vitamin B₃) is another effective treatment. Not only does it lower the LDL levels and triglycerides but it also raises the HDL levels.

Combination therapies involving mixtures of statins and niacin have been described. Advicor® is one such combination drug containing lovastatin and niacin. Research indicates that the treatment of individuals with high cholesterol is more effective by combining the statins and niacin than by each of them individually.

There is a need for better therapies for treating high blood cholesterol and related disorders owing to the prevalence of such disorders among the adult population. The combination of a statin such as mevinolin, niacin and ginkgo allows an improved distribution of drugs throughout the body including narrow blood capillaries. Thus, this novel combination therapy offers a significant improvement over currently available therapies.

### SUMMARY OF THE INVENTION

The present invention provides compositions and convenient methods for the delivery of a statin such as mevinolin, niacin and ginkgo compositions to patients with high blood cholesterol.

In one aspect, the present invention provides a pharmaceutical composition comprising an effective amount of a mixture of a statin such as mevinolin, niacin and ginkgo and a pharmaceutically acceptable carrier.

In another aspect, the present invention provides a pharmaceutical composition where the composition is in the form of a gel, a sublingual lozenge, a solution suitable for aerosolization for mucosal, nasal or pulmonary administration, a liposomal formulation, fine particles in a form suitable for aerosolization for mucosal or pulmonary administration or a tablet.

In one embodiment, the pharmaceutical composition in the form of a gel is disposed within a capsule.

In a particular embodiment, the composition comprises 20-40 mg of a statin, 500-1000 mg of a suitable form of niacin, and 120-240 mg of a suitable ginkgo biloba extract.

In a further aspect, the present invention provides a method of treating or ameliorating a disease associated with high blood cholesterol, comprising administering to a subject suffering from or suspected of suffering from said disease an effective amount of any of the compositions described above.

In yet another aspect, the present invention provides a method of treating high cholesterol levels in a subject comprising administering to said subject an effective amount of any of the compositions defined above.

In another aspect, the present invention provides a kit comprising a pharmaceutical composition comprising an effective amount of a mixture of mevinolin, niacin and ginkgo and a pharmaceutically acceptable carrier.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

Definitions

The following terms used in the specification and claims shall have the following meanings for the purpose of the Application.

By “active ingredients or compounds” of the invention are meant the types of statins including, but not limited to mevinolin, lovastatin, etc., niacin or inositol hexanicotinate and ginkgo biloba. The active ingredients are used in different mixtures containing varying effective amounts of each active compound of the invention, which would be suitable for the treatment of different types of high blood cholesterol.

By “mixture” is meant a combination containing different types of active ingredients defined above, each in effective amounts, useful for the treatment of high blood cholesterol.

By “effective amount” is meant that amount, which when administered, either alone or in combination, is sufficient to effect the treatment of a condition with high blood cholesterol.

By “inert ingredients” is meant components such as pharmaceutically acceptable carriers, adjuvant, diluents or excipients, etc., that are compatible with the active ingredients of the formulation and that are not deleterious to the recipient thereof.

The term “composition” or a “formulation” as used herein is intended to encompass a product comprising the specified active ingredients in the specified amounts, as well as any product which results, directly or indirectly, from the combination of the specified active ingredients in the specified amounts. Such term is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active compounds of the present invention and a pharmaceutically acceptable carrier.

The terms “administration of” and or “administering a” compound should be understood to mean providing the active compounds of the invention, in any formulation, to an individual in need of treatment.

Compositions and methods are provided that are useful in the treatment of treating or ameliorating a disease associated with high cholesterol. The compositions contain at least three active components: a statin, such as mevinolin, or a statin-containing extract of red yeast rice; niacin; and ginkgo. The nature of the ingredients is described in more detail below. The active ingredients are mixed and instilled in a pharmaceutically acceptable carrier formulation or matrix. Suitable formulations or matrices include, but are not limited to, controlled release tablets, hard or soft capsules, pressed pills, gel caps, dispersible powders or granules, emulsions, and the like. Methods of preparing suitable formulations or matrices are well known in the art. These formulations or matrices are patient-friendly, and permit self-administration of effective amounts of the active compounds. The invention thereby minimizes inconvenience and discomfort for the patient and alleviates the burden and time demands imposed on medical staff.

The compositions of the invention are useful in a method for the prevention or treatment of high blood cholesterol disorders in certain combinations. The specific weight ratio of the respective ingredients in the compositions may be varied when necessary and will depend upon the effective dose of each ingredient or the effective dose of the combination of all the active ingredients in a formulation. Generally, an effective dose of each will be used.

The skilled artisan will appreciate that the combination of active ingredients also may be administered separately in the methods of the invention unless specifically indicated otherwise. In addition, active ingredients may be administered in any order and in any subcombination.

Further, compositions of the present invention may be used in combination with other compositions that are used in the treatment/prevention/suppression or amelioration of high blood cholesterol and related disorders. Such other drugs may be administered, by a route and in an amount commonly used therefore, contemporaneously or sequentially with a compound of the present invention. When a composition of the present invention is used contemporaneously with one or more other drugs or herbal supplements, vitamin supplements, etc., a pharmaceutical composition may be used that contains the other drugs in addition to the composition of the present invention. Accordingly, the compositions of the present invention include formulations or matrices that contain one or more other active ingredients, in addition to the compositions of the present invention.

In another embodiment, the present invention provides a kit comprising a composition or formulation of the invention and instructions for storage, administration, dosing, disease state for which the formulation is useful, etc. In yet another embodiment, the present invention provides an article of manufacture comprising a composition or formulation of the invention and an apparatus to dispense or administer the formulation to a given patient, such as container for housing the compound, etc.

Individual Components

Component A

Component A is a statin such as mevinolin or an extract of red yeast rice comprising a known and effective amount of mevinolin. Other statins are known in the art and may be used in place of mevinolin. Extracts of red yeast rice are well known in the art and can be standardized relative to the amount of mevinolin using methods that are well known in the art. An effective amount is any amount in the range of 1-500 mg, although a typical daily dose of mevinolin is 20-40 mg per patient per day.

Component B

Component B is an effective amount of niacin or any of its other pharmaceutically acceptable forms such as inositol hexanicotinate or flush-free niacin. An effective amount is any amount in the range of 1-2000 mg, although a typical dosage is in the range of 500-1000 mg.

Component C

Component C is an effective amount of an herbal extract of ginkgo biloba. Such extracts are well known in the art and are commercially available from a number of suppliers. A standardized extract is commercially available in Europe as EGb 761. Comparable extracts are available in the United States as Ginkgold, Ginkoba, and Ginkai. Standardized preparations typically contain 24 percent ginkgo flavonoid glycosides, 6 percent terpene lactones, and no more than 5 parts per million ginkgolic acids.

An effective amount is any amount in the range of 1-500 mg, although a typical dosage is 120-240 mg per day in a single dose.

Mixture

A mixture of the above components is prepared by physically mixing any or all of the three components in any order and in any suitable proportion such that the amount of each component is be within the corresponding range as indicated above. A typical formulation contains 20-40 mg component A, 500-1000 mg component B and 120-240 mg of component C, although the skilled artisan will recognize that higher or lower amounts may be used under appropriate circumstances.

Methods of making the Pharmaceutical Compositions

The pharmaceutical compositions of this invention conveniently are presented in dosage unit form and may be prepared by methods that are well known in the art of pharmacy. Suitable methods are described in, for example, Remington, The Science and Practice of Pharmacy, ed. Gennaro et al., 20th Ed. (2000), although the skilled artisan will recognize that other methods are known and are suitable for preparing the compositions. All methods include the step of bringing the active ingredients into association with the carrier which constitutes one or more accessory ingredients. In general, the pharmaceutical compositions are prepared by uniformly and intimately bringing the active ingredients into association with a liquid carrier or a finely divided solid carrier or both, and then, if necessary, shaping the product into the desired formulation. In the pharmaceutical composition the active ingredients are included in an effective amount, discussed above, sufficient to produce the desired effect upon the process or condition of diseases.

The pharmaceutical compositions containing the active ingredients may also be in a form suitable for oral use, for example, as tablets. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredients in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredients are mixed with an inert solid diluent, for example, calcium carbonate, calcium phosphate or kaolin; or as soft gelatin capsules wherein the active ingredients are mixed with water or an oil medium, for example peanut oil, liquid paraffin, or olive oil.

It is an object of the present invention to provide patient-friendly modes of delivery to patients of such effective amounts of the active ingredients without the inconvenience and discomfort of subcutaneous and intramuscular injections.

Methods of Treatment

In accordance with the invention, the mixture is instilled in a carrier matrix, such as controlled release pills, tablets, hard or soft capsules, pressed pills, gel caps, dispersible powders or granules, etc., all for patient-friendly, self-administration of effective amounts of the mixture. The mixture in formulations such as tablets, hard or soft capsules, etc. are preferably absorbed directly via the mucosa, such as buccal, nasal mucosa, into the blood stream before being subjected to digestion and degradation in the liver. Preferred formulations include fast absorbing capsules or tablets, etc.

Thus, the formulations of the present invention may be administered, but are not limited to oral routes of administration and may be formulated, alone or together, in suitable dosage unit formulations containing conventional non-toxic pharmaceutically acceptable carriers, adjuvants and vehicles appropriate for each route of administration.

What is claimed is:

1. A pharmaceutical composition comprising: an effective amount of a statin, a pharmaceutically acceptable form of niacin, and gingko biloba, in an amount effective for lowering serum cholesterol in human subjects, and a pharmaceutically acceptable carrier.

2. The composition according to claim 1 wherein said composition is in the form of a gel.

3. The composition according to claim 2 wherein said gel is disposed within a capsule.

4. The composition according to claim 1 wherein said composition is in the form of a tablet.

5. The composition according to claim 1 wherein said statin is in the form of a red yeast rice extract.

6. The composition according to claim 1 wherein said statin is mevinolin.

7. A composition according to claim 1, wherein said composition comprises 20-40 mg of said statin, 500-1000 mg of said niacin, and 120-240 mg of said ginkgo biloba extract.

8. A method of treating or ameliorating a disease associated with high serum cholesterol, comprising administering to a subject suffering from or suspected of suffering from said disease an effective amount of a composition according to claim 1.
